(19) United States
(12) Patent Application Publication
(10) Pub. No.: US 2017/0085547 A1
(43) Pub. Date: Mar. 23, 2017

(54) STORING, INDEXING AND RECALLING DATA BASED ON BRAIN ACTIVITY

(71) Applicant: INTERNATIONAL BUSINESS MACHINES CORPORATION, ARMONK, NY (US)

(72) Inventors: MURILO GONCALVES De AGUIAR, HORTOLANDIA (BR); SILVIA CRISTINA SARDELA BIANCHI, Sao Paulo (BR); VICTOR FERNANDES CAVALCANTE, HORTOLANDIA (BR); GUILHERME STEINBERGER ELIAS, HORTOLANDIA (BR); MARCOS VINICIUS LANDIVAR PARAISO, HORTOLANDIA (BR); FABIO MINORU TANADA, HORTOLANDIA (BR); SERGIO VARGA, HORTOLANDIA (BR)

(21) Appl. No.: 14/861,729

(22) Filed: Sep. 22, 2015

(51) Int. Cl. H04L 29/06; G06F 17/30; A61B 5/055; A61B 5/04; A61B 5/0476

(52) U.S. Cl. CPC H04L 63/08; A61B 5/04008; A61B 5/0476; A61B 5/055; G06F 17/3033; H04L 63/10

(57) ABSTRACT

In a method for storing and recalling stored data, data to be stored is received and a first brain activity information from a user is received. The first brain activity is hashed to generate a first brain activity information hash value. The data is stored within a database and indexed. The indexing is done according to the first brain activity information hash value. The stored data is recalled when a request to recall the stored data is received along with a second brain activity information from a user. The received second brain activity is hashed to generate a second brain activity information hash value. The second brain activity information hash value is used to identify a location of the stored data, within the database, based on the indexing, by matching the second brain activity information hash value to the first brain activity information hash value. The stored data is then retrieved based on the identified location.

### FIELD OF INVENTION

The present invention relates to a method and system for storing, indexing and recalling data and more specifically storing, indexing and recalling data based on brain activity.

### BACKGROUND

Users are creating a large amount of data every day. This explosion of information is increasing the amount of data stored at an exponential rate. This data is being stored on a plethora of devices and solutions. However, users may have difficulty finding a specific piece of data after a period of time has passed. More effective measures for users to recall their sought after data are desirable.

### SUMMARY

According to an embodiment of the present invention received data is stored and indexed based on a first brain activity information from a user. The received first brain activity is hashed to generate a first brain activity information hash value. The received data to be stored within a database is indexed and stored within the database. The indexing is done according to the first brain activity information hash value. The stored data is recalled when a request to recall the stored data is received along with a second brain activity information from a user. The received second brain activity is hashed to generate a second brain activity information hash value. The second brain activity information hash value is used to identify a location of the stored data, within the database, based on the indexing, by matching the second brain activity information hash value to the first brain activity information hash value. The stored data is then retrieved based on the identified location.

According to an embodiment of a present invention metadata associated with previously stored data is received including the location of the previously stored data. Brain activity information from a first user is received and the received brain activity and metadata is hashed to generate a first brain activity information hash value. The previously stored data is hashed according to the first brain activity information hash value. The stored data is recalled by receiving a set of thought attributes and metadata from a second user. Brain activity information is simulated based on the received set of thought attributes and the simulated brain activity and metadata is hashed to generate a second brain activity information hash value. The location of the stored data is identified by matching the second brain activity information hash value to the first brain activity information hash value. The stored data is retrieved based on the identified location.

According to an embodiment of a present invention including a processor and a non-transitory, tangible, program storage medium, readable by the computer system, embodying a program of instructions executable by the processor to perform method steps for storing and recalling stored data. The data to be stored and the first brain activity information from a user are received. The received first brain activity is hashed to generate a first brain activity information hash value. The received data is stored within a database and indexed, within the database, according to the first brain activity information hash value.

### BRIEF DESCRIPTION OF THE DRAWINGS

The accompanying drawings, which are incorporated herein and form part of the specification, illustrate the present invention and, together with the description, further serve to explain the principles of the invention and to enable a person skilled in the pertinent art to make and use the invention.

FIG. 1 is a schematic block diagram of a user storing and retrieving data including the use of brain activity information.

FIG. 2 is a table of automatically identified metadata including brain activity information.

FIG. 3 is a flowchart illustrating where data is stored, indexed and recalled using brain activity information.

FIG. 4a is a flowchart illustrating where data is stored and indexed using brain activity information.

FIG. 4b is a flowchart illustrating where data is recalled using brain activity information.

FIG. 5a is a flowchart illustrating where data is stored and indexed using brain activity information and environment sensor data.

FIG. 5b is a flowchart illustrating where data is recalled using brain activity information and environment sensor data.

FIG. 6a is a flowchart illustrating where data is stored and indexed using brain activity information and a sensitivity level is determined based on the brain activity information.

FIG. 6b is a flowchart illustrating where data is recalled using brain activity information and a user's credentials are verified if required by the sensitivity level.

FIG. 7a is a flowchart illustrating where data is stored and indexed using brain activity information and there is a check to determine if there is sufficient information to recover the data.

FIG. 7b is a flowchart illustrating where data is recalled using brain activity information and there is a check to determine if there is sufficient information to recover the data.

FIG. 8 is a schematic block diagram illustrating a system for storing, indexing and recalling data using brain activity information and environmental information.

FIG. 9 is a schematic block diagram illustrating a computer system.

### DETAILED DESCRIPTION

In one approach to indexing, a user manually enters metadata relating to data into an indexing system such as a file name, file directory location and tags. A user may then recall this information related to the data at a later date, perhaps years later, to retrieve the data.

That approach to indexing systems and methods does not allow a user to locate the relevant digital information without a combination of features or attributes or keys from the user's memory or other sources. That indexing system and method does not facilitate a user retrieving data if the user does not remember the identifying features or attributes of the data. A user who fails to recall these identifying features or attributes effectively loses their data without hope of recovering the information.

An exemplary embodiment of the invention is directed to an indexing system and method that uses brain activity information combined with other environmental information to store and recover data. In accordance with the exemplary embodiment, a brain computer interface BCI, in association with an analytics system, is leveraged to associate data with a subconscious image and or an emotional response derived from events associated with the user actions. The brain computer interface BCI in association with an analytics system indexes the brain activity data with the events associated with the user actions independently of a hard file indexing methodology, physical database indexing or storage policy. Such a system and method will receive input including imagining, thinking, talking, writing, performing a physical movement or the like and match the input with the stored attributes in order to retrieve the desired data.

In exemplary embodiments of the invention, the indexing system may optionally include other sensors such as a camera, GPS, physiological, accelerometer, physiological, barometer and/or a gyroscope to provide additional information to the indexing system.

Various attributes of the data may be used to index the data. The attributes may include context, relationships with other data and/or information, timestamp, data name, image summary, relevant text, content summary, file name, storage path and the like.

In exemplary embodiments of the invention, the indexing system may optionally select some or all of the attributes of the data. The selected attributes may be hashed and stored in an index to aid in later retrieving the data.

If desired, the indexing system may optionally include attributes that define the security level needed for the data, restricting the data to a specific group.

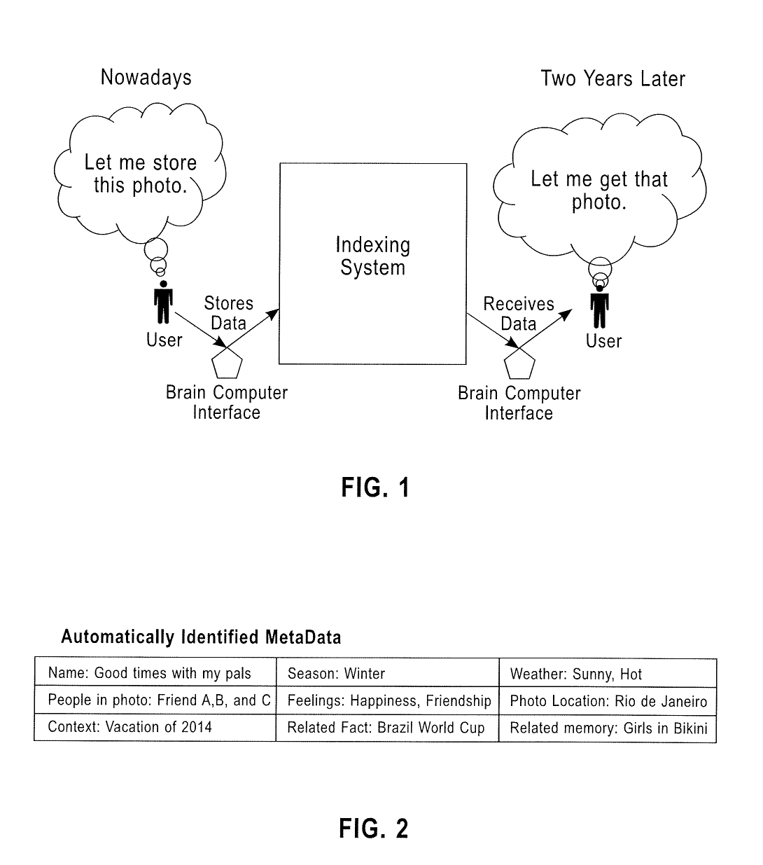

FIG. 1 depicts an embodiment of the invention illustrating the BCI 102. The BCI 102 captures signals from the human neural implicit network of a user 101 combined with an analytics system. For example, the BCI 102 may detect brain activity from a user's 101 brain when a photograph is taken. A brain analyzer may interpret the brain activity or part of the brain activity as an attribute such as “happiness and friendship” and store that information as metadata. Other types of information may also be collected from other sensors when the photograph is taken.

The indexing system 103 may also analyze the data and detect one or more context attributes from the content of the data. In the example of the photograph the indexing system 103 may detect attributes such as the identity of people in the photograph by using facial recognition. The indexing system 103 may also receive attributes from environmental sensors when the data is created. The user 101 may also manually input attributes associated with the data. The indexing system 103 may associate these various attributes with the data and store the attributes in an index.

FIG. 2 shows the Automatically Identified Metadata used in the indexing system to identify the stored data. The Automatically Identified Metadata may store the attribute received from the BCI. The indexing system may also determine a related memory from the information obtained from the BCI and the related memories may be stored in the Automatically Identified Metadata. This Automatically Identified Metadata may include information that identifies the data specifically such as the name of the data. The Automatically Identified Metadata may also include the identities of people in the data if they can be identified or the identities of people associated with creating the data. The people in the data may be identified using facial recognition if the data is a photograph or voice recognition in the case of a sound recording. The Automatically Identified Metadata may also store attributes determined from general information around the creation of the data such as the context, the season and a related fact about the data. The Automatically Identified Metadata may also include attributes associated with environmental data and may include the weather when the data was created and the location where the data was created.

FIG. 1 also shows a similar process that is used by the indexing system 103 to recall desired data. The BCI 102 captures signals from the human neural implicit network of a user combined with an analytics system. For example, the BCI 102 may detect brain activity from a user's 101 brain about a photograph the user 101 desires to recall. The indexing system 103 may also receive other context attributes from the user 101. The indexing system 103 uses these attributes to search the index for the relevant photograph.

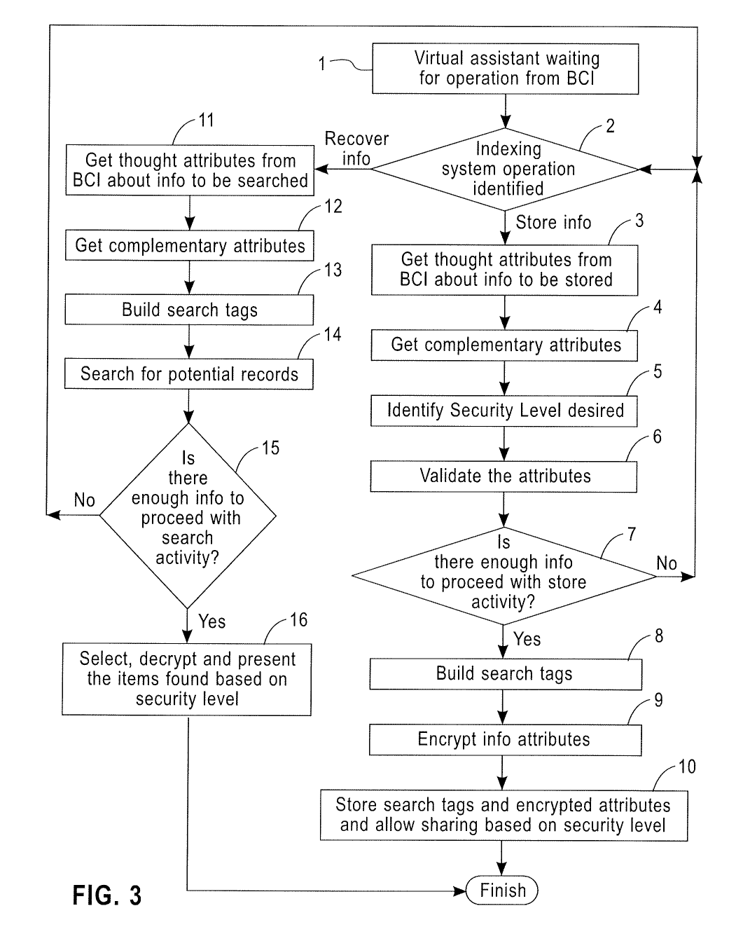

FIG. 3 depicts an embodiment comprising an indexing method for storing and recalling information. The virtual assistant begins in a stand-by mode waiting for user input from the BCI (S1). The virtual assistant activates upon receiving the user input and transmits the user input to the indexing system. The indexing system determines if the user seeks to store or recover data. Where the data may be either static, dynamic, metadata representing a data or a metadata representing a non-digital entity. The determination is conducted by associating brain activity data with storing or recovering. The indexing system may also receive indications to store or recover from other sensors including a keyboard, camera, GPS, physiological, accelerometer, physiological, barometer and/or a gyroscope. The data to be stored may be a photograph, a video, a sound file, any kind of digital data or metadata associated with the data to be stored. The metadata may also refer to data stored in other locations not associated with the indexing system or data that cannot be stored digitally. The metadata about the data already stored in other locations or metadata to locate data that cannot be stored digitally may come from other indexing systems, location systems, GPS, from the user's interaction with the indexing system, from files in the user's workstation, mobile data or any other repository of data. The indexing system performs the next step based on the determination (S2).

If the user desires to store data the indexing system will read the user's thoughts and/or images by activating the BCI and capturing the digital output from the brain activity reading (S3). The user's brain activity may be constantly monitored and the brain activity reading may be a portion of the continuously received brain activity. The BCI device is used to detect a user's brain patterns, such as an electroencephalography EEG, functional magnetic resonance imaging FMRI, magnetoencephalography MEG or similar, in response to stimuli and record the result. A brain analyzer, in the indexing system, generates one or more attributes that can be used as input for tagging the data to be stored from the brain activity output from the BCI.

The indexing system includes one or more other sensors in addition to the BCI. These other sensors are used to capture additional complementary attributes (S4). These additional complementary attributes may be used to augment the one or more attributes generated from the digital output from the brain activity reading and/or attribute data from other sensors. The complementary attributes may include attributes manually entered by the user, context attributes determined from the data itself, environmental attributes and other types of attributes.

The virtual assistant may receive input from a user about the data. The user may manually enter identity attributes including context, relationship with other data/information, timestamp, data name, image summary, relevant text, content summary, file name, storage path. The indexing system may also automatically generate context attributes from the data. For example, the indexing system may use facial recognition to identify users in a photograph or determine background information from the data. Environmental data may include the weather when the data was created and the location when the data was created. The indexing system may retrieve the environmental data from various environmental sensors including a thermostat, barometer, radar and or satellite imaging. The BCI may also generate environmental data from the patterns of the user's brain activity.

The indexing system may include other sensors including a camera, microphone, clock for determining a time stamp, keyboard, mouse, touch screen, asset name, mouse, accelerometer, barometer and/or a gyroscope relationship. Different kinds of devices or programs may be used by the indexing system to identify the context attributes of the data such as brain analyzers, sound analyzers, image/video analyzers, NLP and/or similar sensors. The context attributes may include the name of the file, the name of subjects in the file, the topic of the data, the season when the data was created, the feeling of the user, the weather at the time the data was created, the physical location where the data was created, miscellaneous facts related to the data and/or miscellaneous memories related to the data.

The indexing system may identify the desired security level for data based on the information received from the user (S5). The indexing system may determine the security level of data by a plurality of methods. In an exemplary embodiment the brain analyzer identifies brain activity information associated with the security level of the data. The brain activity information may indicate that the security level of the data should be maximum security, private, public, restricted to a location, restricted to a group, restricted to few users and/or any other classification that would limit access to the data. The security level may indicate the user or group of users that have access to the data, the type of encryption used, the number and type of credentials used and the type of facility that may store the data. The indexing system stores the user data into the corresponding data space, with the required security level. The indexing system may recall the data presenting the data according to the security level set when the data was stored.

In an embodiment of the invention, the user may request a security level through the virtual assistant. Using an interface of the virtual assistant, the user may select a security level for the data. The user may select a security level from a list including maximum security, private, public, restricted to a location, restricted to a group, restricted to few users and/or any other classification that would limit access to the data. The user may also enter a customized security level. The indexing system stores the user data into the corresponding data space, with the required security level. The indexing system may recall the data presenting it according to the security level set when it was stored.

An undesirable situation may occur where an insufficient number of attributes may be stored to allow for an effective retrieval of a data (S6). To prevent this possibility the indexing system performs a consistency check. The consistency check validates that the attributes are of a sufficient number and consistent for later recovering the relevant information. The indexing system includes a feedback mechanism via a virtual assistant component where the user can modify, add or remove information and attributes in order to refine the metadata. If there are not enough attributes associated with the data to uniquely identify the data the archive system will further examine the data to discern additional attributes (S7).

Entries for the index are created based on the attributes received from the BCI and other sensors (S8). The indexing system receives attributes and collects this information into search tags. The search tag may store attributes including attributes from the BCI, attributes manually entered by the user, context attributes determined from the data itself, environmental attributes and other types of attributes. The search tags may also store information regarding the security level. Through the virtual assistant, the user can dynamically add, change or remove attributes in order to list less or more potential items related to relevant information about the data. This feedback may be used by the indexing system to refine future search keys generated during future storage phases.

The virtual assistant component may be used to interact with the user's request where the user can add or remove attributes from an encrypted tag and store the encrypted tag with the data (S9). In an embodiment of the invention the indexing system may receive attributes from several sources including the BCI, environmental sensors and the virtual assistant. Some of these attributes may be sensitive and it may be desirable for the user to store the sensitive attributes. The indexing system may designate an attribute as sensitive based on the information contained within the attribute. Additionally, the user may manually designate attributes as sensitive using the virtual assistant. The indexing system encrypts the sensitive attribute and stores the encrypted sensitive attribute with the data in the database and/or with the search tag in the search tag database.

The indexing system then stores the data according to the desired security level (S10). The indexing system may also generate one or more hashes from the attributes received from the user. These hashes are stored in an index and used by the indexing system to identify data. The indexing system may hash the attributes using a hash function such as MD5, SHA-1, SHA-512, SHA-3 or any other hash functions.

FIG. 3 also depicts an embodiment comprising an indexing method for retrieving information. If the user desires to recover the data the indexing system will read the user's brain activity by activating the BCI interface and capturing the digital output from the brain activity reading (S11). The user's brain activity may be constantly monitored and the brain activity reading may be a portion of the continuously received brain activity. During the acquisition of the user's brain activity to recall the data the user may be engaged in thinking about the circumstances in which the data to be stored was generated or stored. These circumstances may include the user being engaged in talking about, writing about, or engaging in a physical gesture related to the circumstances in which the data to be stored was generated or stored. The BCI device is used to detect a user's brain patterns, such as an electroencephalography EEG, functional magnetic resonance imaging FMRI, magnetoencephalography MEG or similar, in response to stimuli and record the result. A brain analyzer, in the indexing system, generates one or more attributes that can be used as input for tagging the data to be stored from the brain activity output from the BCI.

The indexing system receives context data related to the data. This context data is used to complement the attribute data captured by the BCI (S12). These additional complementary attributes may be used to augment the one or more attributes generated from the digital output from the brain activity reading and/or attribute data from other sensors. The complementary attributes may include attributes manually entered by the user, context attributes determined from the data itself, environmental attributes and other types of attributes. The virtual assistant may receive input from a user about the data. The user may manually enter identity attributes including context, relationship with other data/information, timestamp, data name, image summary, relevant text, content summary, file name, storage path. The indexing system may also automatically generate context attributes from the data. For example, the indexing system may use facial recognition to identify users in a photograph or determine background information from the data. Environmental data may include the weather when the data was created and the location where the data was created. The BCI may also generate environmental data from the user's brain activity.

The indexing system creates search tags based on the attributes received from the BCI and other sensors (S13). The indexing system receives attributes and collects this information into search tags. The search tag may store attributes including attributes from the BCI, attributes manually entered by the user, context attributes determined from the data itself, environmental attributes and other types of attributes. Through the virtual assistant, the user can dynamically add, change or remove attributes in order to list less or more potential items related to relevant information about the data. This feedback may be used by the indexing system to refine future search keys generated during future storage phases. The Indexing system may hash the search tag.

The recreated search tag may be used to search the index (S14). The indexing system may use a hash derived from the search tag to search the index. If the indexing system does not find the requested data then the indexing system attempts to acquire more attributes from the user and reattempt the search (S15).

The indexing system then selects the found data in the database. If the data is restricted based on a security level the user interacts with the virtual assistant to satisfy the security level requirements (S16). When the user tries to recover some data, the system presents a list of the items found and their attributes/search tag. The proposed system can present results to the user based on technologies related to visualization or augmented reality.

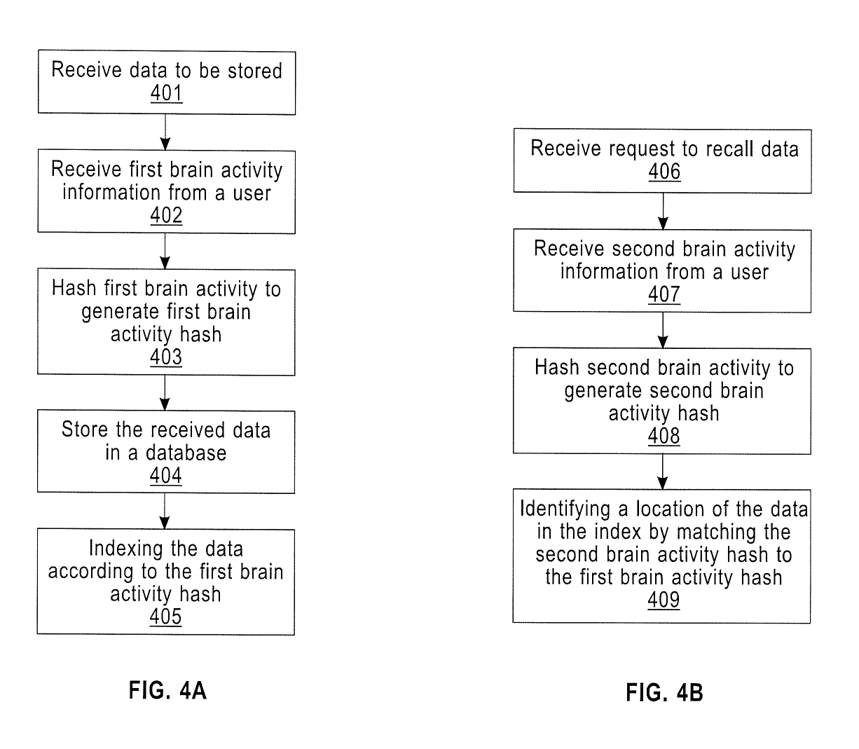

FIG. 4a illustrates an embodiment of the invention describing a method for storing and indexing data. The indexing system receives the data to be stored or metadata about the data already stored in other locations (S401). The indexing system may receive data from the user or another source through one or more interfaces. The user may input the data through a user interface adapter such as a keyboard or mouse or through other types of storage media. The indexing system may also acquire data from a remote server through a network adapter.

The BCI records the user's first brain activity that is associated with the data. A brain analyzer then generates the first brain activity information based on the user's brain activity (S402). The BCI device is used to detect a user's brain patterns, such as an EEG, FMRI, MEG or similar, in response to stimuli and record the result. A brain analyzer, in the indexing system, generates one or more attributes that can be used as input for tagging the data to be stored from the brain activity output from the BCI.

The indexing system hashes the first brain activity information to generate the first brain activity information hash value (S403). The indexing system may generate a search tag from attributes including user's brain patterns captured by the BCI. The indexing system may hash the search tag using a hash function such as MD5, SHA-1, SHA-512, SHA-3 or any other hash functions.

The indexing system stores the received data in a database (S404). The database may be implemented on a local storage device, an external storage device or on a remote cloud storage solution. The data may be transmitted from the indexing system to the local storage via bus or to a remote storage device over the network through a network adapter.

The indexing system stores the data and the associated search tags (S405). The data and the search tags are stored in separate databases. The search tag may point to the location of the stored data.

FIG. 4b illustrates an embodiment of the invention occurring where the user desires to recall the data stored in the storage device. The indexing system receives a request to recall the data (S406). The user may receive a request for data through the BCI or the virtual assistant. For example, the BCI may transmit brain activity to the indexing system. The brain activity would be analyzed to determine if the user desires to request data. Alternatively, the user may input a request to the virtual assistant using a user interface adapter.

In a process similar to the method previously described in FIG. 4a the BCI records the user's second brain activity that is associated with the data. A brain analyzer then generates the second brain activity information based on the user's brain activity (S407). During the acquisition of the user's brain activity to recall the data the user may be engaged in thinking about the circumstances in which the data to be stored was generated or stored. These circumstances may include the user being engaged in talking about, writing about, or engaging in a physical gesture related to the circumstances in which the data to be stored was generated or stored. The BCI device is used to detect a user's brain patterns, such as an EEG, FMRI, MEG or similar, in response to stimuli and record the result. A brain analyzer, in the indexing system, generates one or more attributes that can be used as input for tagging the data to be stored from the brain activity output from the BCI.

The indexing system hashes the second brain activity information to generate the second brain activity information hash value (S408). The indexing system may generate a search tag from attributes including user's brain patterns captured by the BCI. The indexing system may hash the search tag using a hash function such as MD5, SHA-1, SHA-512, SHA-3 or any other hash functions.

The indexing system searches the index with the second brain activity information hash value until it finds a matching entry (S409). With the matching index information the indexing system may recall the desired data and present the data to the user or another desired entity.

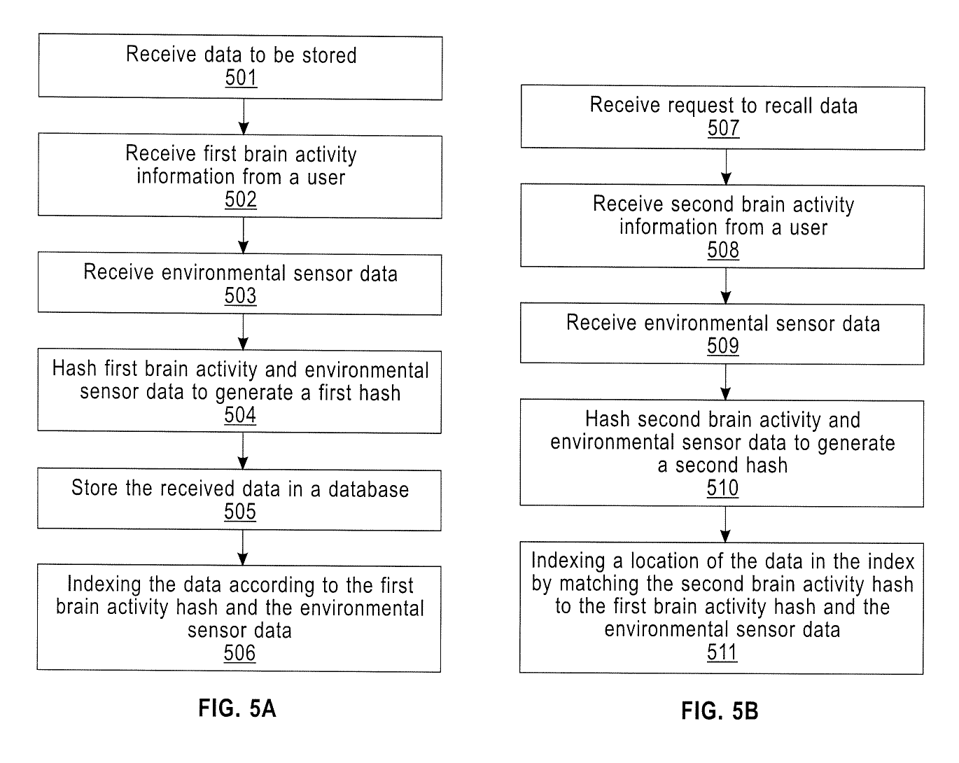

FIG. 5a illustrates an embodiment of the invention describing a method for storing and indexing data. The indexing system receives the data to be stored (S501). The indexing system may receive data from the user or another source through one or more interfaces. The user may input the data through a user interface adapter such as a keyboard or mouse or through other types of storage media. The indexing system may also acquire data from a remote server through a network adapter.

The BCI records the user's first brain activity that is associated with the data. A brain analyzer then generates the first brain activity information based on the user's brain activity (S502). The BCI device is used to detect a user's brain patterns, such as an EEG, FMRI, MEG or similar, in response to stimuli and record the result. A brain analyzer, in the indexing system, generates one or more attributes that can be used as input for tagging the data to be stored from the brain activity output from the BCI.

The indexing system may receive environmental information about the data from one or more different environmental sensors. The environmental sensors may include a camera, physiological, GPS, physiological, accelerometer, barometer, gyroscope and/or other sensors. The BCI may also generate environmental data from the user's brain activity. The environmental sensors may generate environmental sensor data including location, sound, weather, height, physical movement, stationary heart beat and/or moving heart beat (S503).

The indexing system hashes the first brain activity information and the environmental sensor data to generate a first hash value (S504). The indexing system may generate a search tag from attributes including user's brain patterns captured by the BCI and the environmental sensor data. The indexing system may hash the search tag using a hash function such as MD5, SHA-1, SHA-512, SHA-3 or any other hash functions.

The indexing system stores the received data in a storage device (S505). The database may be implemented on a local storage device, an external storage device or on a remote cloud storage solution. The data may be transmitted from the indexing system to the local storage via bus or to a remote storage device over the network through a network adapter.

The indexing system indexes the data according to the first hash (S506). The data and the search tags are stored in separate databases. The search tag may point to the location of the stored data. The data and the search tags are stored in separate databases. The search tag may point to the location of the stored data.

FIG. 5b illustrates an embodiment of the invention occurring where the user desires to recall the data stored in the storage device. The indexing system receives a request to recall the data (S507). The user may receive a request for data through the BCI or the virtual assistant. For example, the BCI may transmit brain activity to the indexing system. The brain activity may be analyzed to determine if the user desires to request data. Alternatively, the user may input a request to the virtual assistant using a user interface adapter.

In a process similar to the previous method described in FIG. 5a the BCI records the user's second brain activity that is associated with the data. A brain analyzer then generates the second brain activity information based on the user's brain activity (S508). During the acquisition of the user's brain activity to recall the data the user may be engaged in thinking about the circumstances in which the data to be stored was generated or stored. These circumstances may include the user being engaged in talking about, writing about, or engaging in a physical gesture related to the circumstances in which the data to be stored was generated or stored. The BCI device is used to detect a user's brain patterns, such as an EEG, FMRI, MEG or similar, in response to stimuli and record the result. A brain analyzer, in the indexing system, generates one or more attributes that can be used as input for tagging the data to be stored from the brain activity output from the BCI.

The indexing system receives the environmental sensor data (S509). The environmental sensor data may come from one or more different sources or from a combination of sources. The environmental sensor data may come from the environmental sensors from a device describing current conditions at a location. The environmental sensor data may also be input from a user using the virtual assistant. The BCI may also generate environmental data from the user's brain activity. The environmental sensor data may come from another piece of data stored locally on the device or retrieved from a remote source over a network.

The indexing system hashes the second brain activity information and the environmental sensor data to generate the second hash value (S510). The indexing system may generate a search tag from attributes including user's brain patterns captured by the BCI and the environmental sensor data. The indexing system may hash the search tag using a hash function such as MD5, SHA-1, SHA-512, SHA-3 or any other hash functions.

The indexing system searches the index with the second hash value until it finds a matching entry (S511). With the matching index information the indexing system may recall the desired data and present the data to the user or another desired entity.

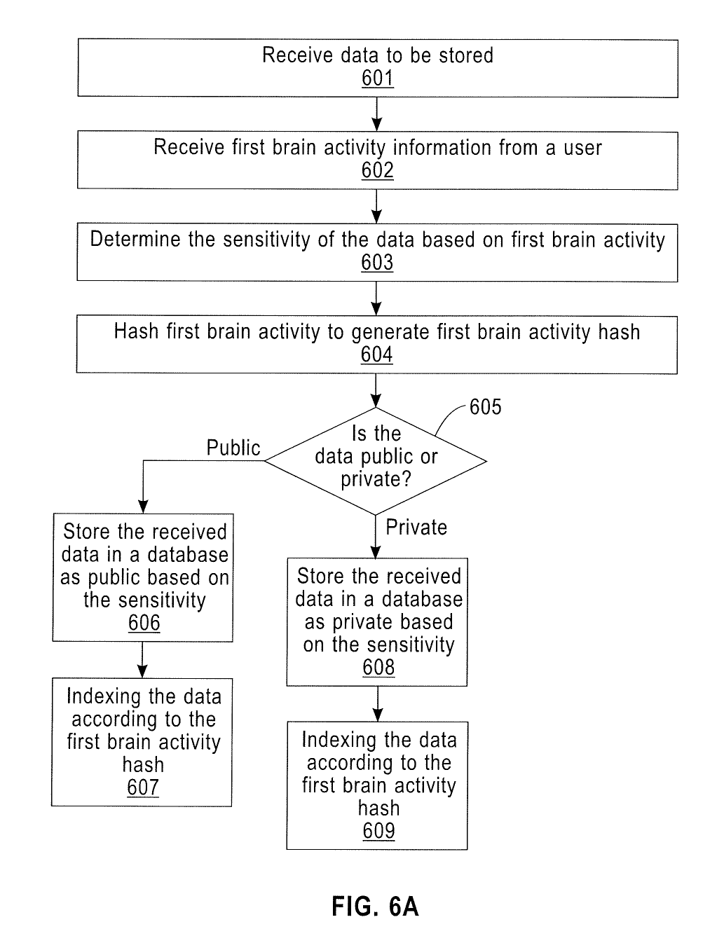

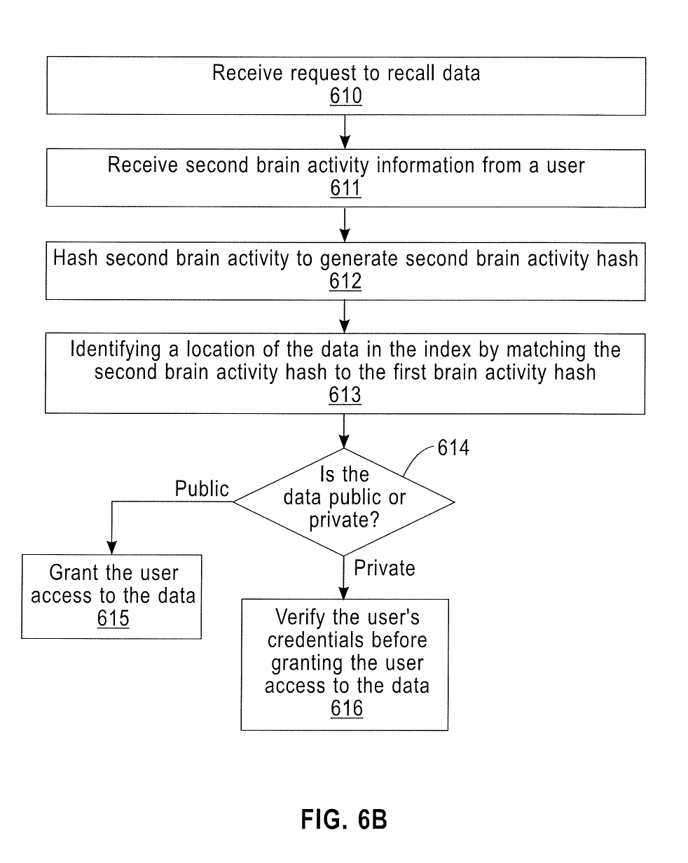

FIG. 6a illustrates an embodiment of the invention describing a method for storing and indexing data. The indexing system receives the data to be stored (S601). The indexing system may receive data from the user or another source through one or more interfaces. The user may input the data through a user interface adapter such as a keyboard or mouse or through other types of storage media. The indexing system may also acquire data from a remote server through a network adapter.

The BCI records the user's first brain activity that is associated with the data. A brain analyzer then generates the first brain activity information based on the user's brain activity (S602). The BCI device is used to detect a user's brain patterns, such as an EEG, FMRI, MEG or similar, in response to stimuli and record the result. A brain analyzer, in the indexing system, generates one or more attributes that can be used as input for tagging the data to be stored from the brain activity output from the BCI.

The indexing system may determine a user's desired security level (S603). This determination may be based on the first brain activity received from the BCI, the environmental sensor data and/or complementary attributes received from other sensors. In FIG. 6a the indexing system determines if the security level of the data will be classified as public or private. The indexing system may also classify the data as maximum security, public, restricted to a location, restricted to a group, restricted to few users or other types of security classifications. The security level may indicate the user or group of users that have access to the data, the type of encryption used, the number and type of credentials used and the type of facility that may store the data. The security level designation may be stored in the search tag and or with the data.

The indexing system hashes the first brain activity information to generate the first brain activity information hash value (S604). The indexing system may generate a search tag from attributes including user's brain patterns captured by the BCI. The indexing system may hash the search tag using a hash function such as MD5, SHA-1, SHA-512, SHA-3 or any other hash functions.

The indexing system determines based on the user's desired security level whether to store the data as public or private (S605). The indexing system checks the designation in the search tag or the data as public or private.

If the data is determined to be public the indexing system stores the received data in a public storage space in a storage device (S606). The database may be implemented on a local storage device, an external storage device or on a remote cloud storage solution. The data may be transmitted from the indexing system to the local storage via bus or to a remote storage device over the network through a network adapter.

The indexing system indexes the data according to the first brain activity hash (S607). The data and the search tags are stored in separate databases. The search tag may point to the location of the stored data.

If the data is determined to be private the indexing system stores the received data in a private storage space in a storage device (S608). The private storage may be encrypted and restrict access to the data to authorized users. The database may be implemented on a local storage device, an external storage device or on a remote cloud storage solution. The data may be transmitted from the indexing system to the local storage via bus or to a remote storage device over the network through a network adapter.

The indexing system indexes the data according to the first brain activity hash (S609). The data and the search tags are stored in separate databases. The search tag may point to the location of the stored data.

FIG. 6b illustrates an embodiment of the invention occurring where the user desires to recall the data stored in the storage device. The indexing system receives a request to recall the data (S610). The user may receive a request for data through the BCI or the virtual assistant. For example, the BCI may transmit brain activity to the indexing system. The brain activity may be analyzed to determine if the user desires to request data. Alternatively, the user may input a request to the virtual assistant using a user interface adapter.

In a process similar to the previous method described in FIG. 6a the BCI records the user's second brain activity that is associated with the data. A brain analyzer then generates the second brain activity information based on the user's brain activity (S611). During the acquisition of the user's brain activity to recall the data the user may be engaged in thinking about the circumstances in which the data to be stored was generated or stored. These circumstances may include the user being engaged in talking about, writing about, or engaging in a physical gesture related to the circumstances in which the data to be stored was generated or stored. The BCI device is used to detect a user's brain patterns, such as an EEG, FMRI, MEG or similar, in response to stimuli and record the result. A brain analyzer, in the indexing system, generates one or more attributes that can be used as input for tagging the data to be stored from the brain activity output from the BCI.

The indexing system hashes the second brain activity information to generate the second brain activity information hash value (S612). The indexing system may generate a search tag from attributes including user's brain patterns captured by the BCI. The indexing system may hash the search tag using a hash function such as MD5, SHA-1, SHA-512, SHA-3 or any other hash functions.

The indexing system searches the index with the second brain activity information hash value until it finds a matching entry (S613). With the matching index information the indexing system may recall the desired data and present the data to the user or another desired entity.

The indexing system locates desired data and determines if the security level is public or private (S614). The indexing system checks the search tag and/or the data for the assigned security level. If the indexing system determines that the data is private the user may transmit the user's credentials to the indexing system.

If the indexing system determines that the data is public the indexing system grants the user access to the data (S615). With the matching index information the indexing system may recall the desired data and present the data to the user or another desired entity.

If the indexing system determines that the user's credentials are correct the indexing system will grant the user access to the data (S616). The user may transmit credentials as brain activity information captured by the BCI. The credentials may also be received by other sensors including environmental sensors and user input adapters through the virtual assistant.

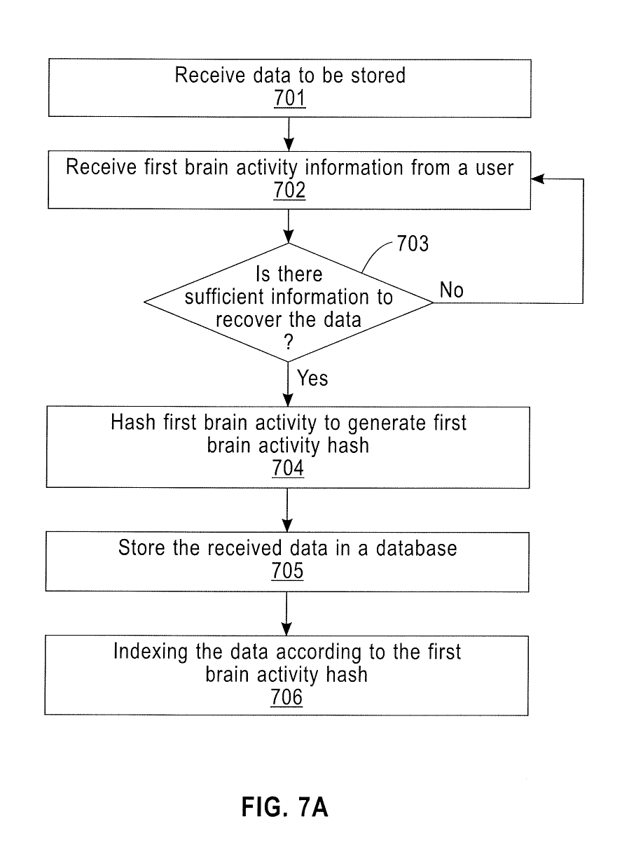

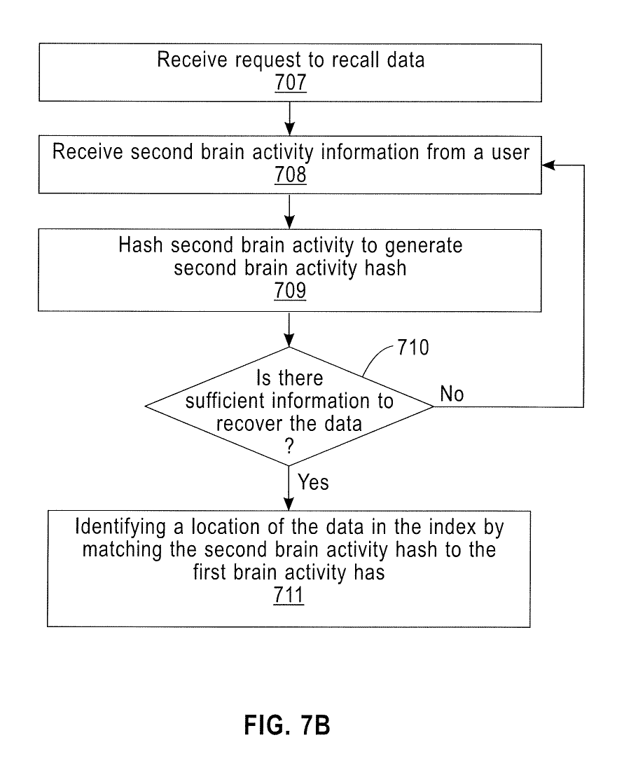

FIG. 7a illustrates an embodiment of the invention describing a method for storing and indexing data. The indexing system receives the data to be stored (S701). The indexing system may receive data from the user or another source through one or more interfaces. The user may input the data through a user interface adapter such as a keyboard or mouse or through other types of storage media. The indexing system may also acquire data from a remote server through a network adapter.

The BCI records the user's first brain activity that is associated with the data. A brain analyzer then generates the first brain activity information based on the user's brain activity (S702). The BCI device is used to detect a user's brain patterns, such as an EEG, FMRI, MEG or similar, in response to stimuli and record the result. A brain analyzer, in the indexing system, generates one or more attributes that can be used as input for tagging the data to be stored from the brain activity output from the BCI.

The indexing system will periodically perform a consistency check. The indexing system performs the consistency check to determine if there are sufficient attributes to recover the data (S703). If there are sufficient attributes the process proceeds to hash the attributes. If there are insufficient attributes to identify the data the indexing system attempts to retrieve more attributes to identify the data. The indexing system may compare the number of attributes contained in the search key with a threshold value or a standard based on the other entries in the index. Standard based on the other entries in the index may be the mean, medium, mode or other similar measure.

The indexing system hashes the first brain activity information to generate the first brain activity information hash value (S704). The indexing system may generate a search tag from attributes including user's brain patterns captured by the BCI. The indexing system may hash the search tag using a hash function such as MD5, SHA-1, SHA-512, SHA-3 or any other hash functions.

The indexing system stores the received data in a storage device (S705). The database may be implemented on a local storage device, an external storage device or on a remote cloud storage solution. The data may be transmitted from the indexing system to the local storage via bus or to a remote storage device over the network through a network adapter.

The indexing system indexes the data according to the first brain activity hash (S706). The data and the search tags are stored in separate databases. The search tag may point to the location of the stored data.

FIG. 7b illustrates an embodiment of the invention occurring where the user desires to recall the data stored in the storage device. The indexing system receives a request to recall the data (S707). The user may receive a request for data through the BCI or the virtual assistant. For example, the BCI may transmit brain activity to the indexing system. The brain activity may be analyzed to determine if the user desires to request data. Alternatively, the user may input a request to the virtual assistant using a user interface adapter.

In a process similar to the previous method described in FIG. 4a the BCI records the user's second brain activity that is associated with the data. A brain analyzer then generates the second brain activity information based on the user's brain activity (S708). During the acquisition of the user's brain activity to recall the data the user may be engaged in thinking about the circumstances in which the data to be stored was generated or stored. These circumstances may include the user being engaged in talking about, writing about, or engaging in a physical gesture related to the circumstances in which the data to be stored was generated or stored. The BCI device is used to detect a user's brain patterns, such as an EEG, FMRI, MEG or similar, in response to stimuli and record the result. A brain analyzer, in the indexing system, generates one or more attributes that can be used as input for tagging the data to be stored from the brain activity output from the BCI.

The indexing system hashes the second brain activity information to generate the second brain activity information hash value (S709). The indexing system may generate a search tag from attributes including user's brain patterns captured by the BCI. The indexing system may hash the search tag using a hash function such as MD5, SHA-1, SHA-512, SHA-3 or any other hash functions.

The indexing system performs the consistency check to determine that there are sufficient attributes to recover the data (S710). If there are sufficient attributes the process proceeds to recover the data. If there are insufficient attributes to identify the data the indexing system attempts to retrieve more attributes to identify the data. The indexing system may compare the number of attributes contained in the search key with a threshold value or a standard based on the other entries in the index. Standard based on the other entries in the index may be the mean, medium, mode or other similar measure.

The indexing system searches the index with the second brain activity information hash value until it finds a matching entry (S711). With the matching index information the indexing system may recall the desired data and present the data to the user or another desired entity.

Other embodiments have been describes in terms of a single user that stores and retrieves the data. In an embodiment of the invention, a different and/or second user may recall the data from the indexing system. In this embodiment a first user inputs the data into the indexing system and using the BCI inputs the brain activity information used to index the data. The indexing system may store in data in the database. A second user may use the BCI to input the thought attributes needed to recall the data into the indexing system. The indexing system may then simulate the brain activity information to generate a second brain activity information hash value. The indexing system may use the second brain activity information hash value to locate the location of the stored data in the database. If the data is subject to a security level the indexing system may require the second user to input a credential and verify the credential before granting the second user access to the secured data.

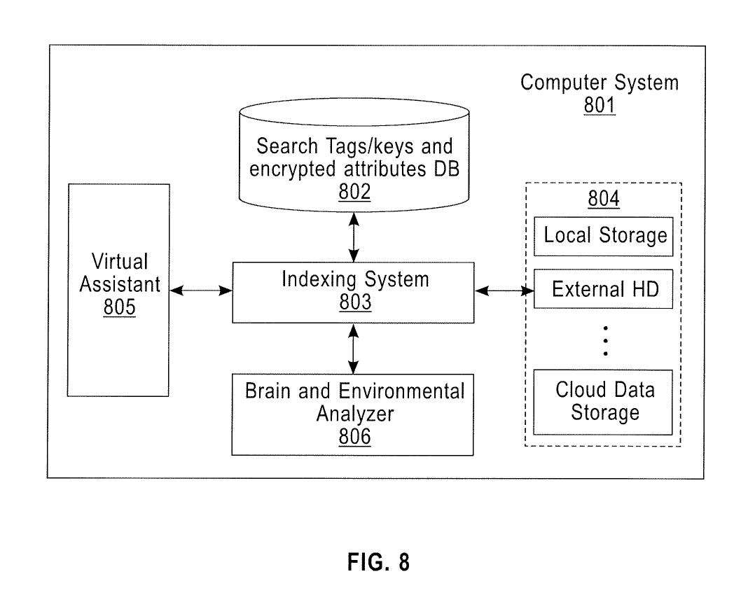

FIG. 8 discloses a computing system 801 for implementing the indexing system 803. The indexing system 803 may be implemented in software, hardware or a combination of software and hardware. The indexing system 803 may be implemented on a desktop, mobile device or in a cloud based computing environment. The virtual assistant 805 may be implemented on various devices including desktops, kiosks and mobile devices where a mobile device may include mobile phones, tablets, laptops and other types of devices. The virtual assistant may have a graphical user interface or another module for transmitting user input to the indexing system. The brain and environment analyzer 806 may receive the brain activity information and the environmental sensor information. The brain and environment analyzer 806 may be implemented in software, hardware or a combination of software and hardware. The brain and environment analyzer 806 interprets the information and generates attributes. These attributes are transmitted to the indexing system 803 and used to identify the data. The indexing system 803 may collect the brain activity information, the environmental sensor data and other information into collections of metadata associated with the data. The collection of metadata may be referred to as a search tag or a search key and may be stored in a database 802 in addition to other types of metadata associated with the data. The indexing system may store the data in a separate database 804 from the metadata. The database 804 may be stored on a local storage device, an external storage device or on a remote cloud storage solution.

An embodiment of the invention includes training the system to recognize a user's different brain activity. A device is used to detect a user's brain activity, such as an MRI, EEG or similar, in response to stimuli and record the result. A brain analyzer may be trained to associate a specific brain activity with a specific stimulus. The stimulus may be an emotion, concept or related to a type of environmental stimulus the user is experiencing, such as nearby people or objects or the temperature. For example, each time a user feels a specific emotion, such as “happiness”, the user notifies the brain analyzer that the user feels happiness, resulting in a learning event. After a plurality of learning events the brain analyzer may be able to associate a specific brain activity with the stimulus, “happiness” in the current example. The brain analyzer may use heuristics or algorithms to distinguish between noise and the brain activity and correctly associate the brain activity with the stimulus.

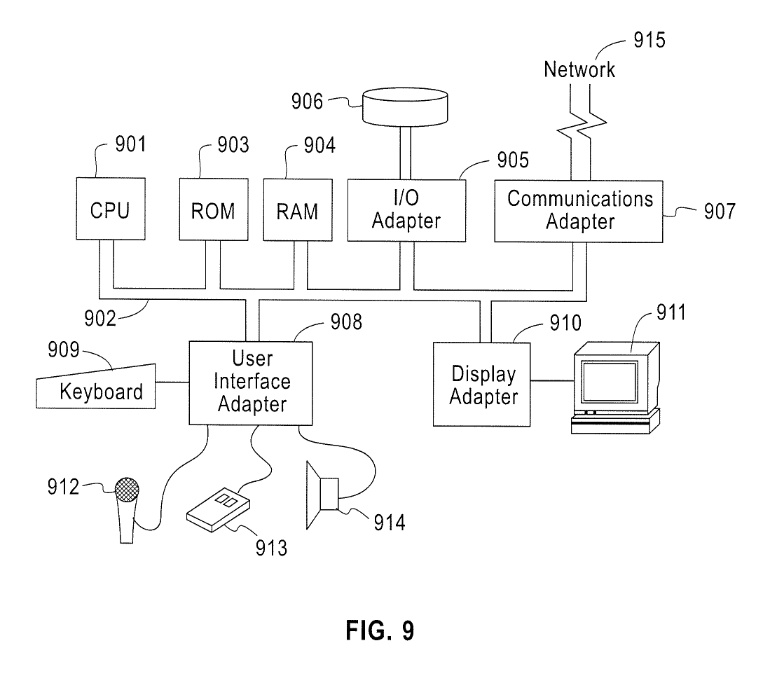

FIG. 9 shows a representative hardware environment associated with a user device and/or server, in accordance with one embodiment. Such figure illustrates a typical hardware configuration of a workstation, mobile device or any other repository of data having a central processing unit 901, such as a microprocessor, and a number of other units interconnected via a system bus 902.

The workstation shown in FIG. 9 includes a Random Access Memory (RAM) 904, Read Only Memory (ROM) 903, an I/O adapter 905 for connecting peripheral devices such as disk storage units 906 to the bus 902, a user interface adapter 908 for connecting a keyboard 909, a mouse 913, a speaker 914, a microphone 912, and/or other user interface devices such as a touch screen and a digital camera (not shown) to the bus 902, communication adapter 907 for connecting the workstation to a communication network 915 (e.g., a data processing network) and a display adapter 910 for connecting the bus 902 to a display device 911. The communication adapter may be a wired or wireless device.

The workstation may have resident thereon an operating system such as the Microsoft Windows® Operating System (OS), a MAC OS, or UNIX operating system. It will be appreciated that a preferred embodiment may also be implemented on platforms and operating systems other than those mentioned. A preferred embodiment may be written using JAVA, XML, C, and/or C++ language, or other programming languages, along with an object oriented programming methodology.

As will be appreciated by one skilled in the art, aspects of the invention may be embodied as a system, method or computer program product. Accordingly, aspects of the invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the invention may take the form of a computer program product embodied in one or more computer readable medium(s) having computer readable program code embodied thereon.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

What is claimed is:

1. A method for storing and recalling stored data, comprising:
receiving data to be stored
receiving first brain activity information from a user;
hashing the received first brain activity to generate a first brain activity information hash value;
storing the received data to be stored within a database;
indexing the stored data, within the database, according to the first brain activity information hash value;
receiving a request to recall the stored data;
receiving second brain activity information from a user;
hashing the received second brain activity to generate a second brain activity information hash value;
identifying a location of the stored data, within the database, based on the indexing, by matching the second brain activity information hash value to the first brain activity information hash value; and
retrieving the stored data based on the identified location.

2. The method of claim 1, wherein, prior to hashing the received first brain activity, environmental sensor data is received, the environmental sensor data and the first brain activity are hashed to generate a first brain activity information hash value and environmental sensor data hash value, the indexing of the stored data is based on both the first brain activity information hash value and the environmental sensor data hash value, and identifying the location of the stored data, within the database, is based on both the matching of the second brain activity information hash value to the first brain activity information hash value and by matching the environmental sensor data hash values.

3. The method of claim 2, wherein matching the environmental sensor data includes interpreting the second brain activity to estimate environmental conditions.

4. The method of claim 2, wherein the environmental sensor data is acquired using a global positioning service (GPS) device, an accelerometer, a barometer, or one or more physiological sensors.

5. The method of claim 1, wherein the data to be stored is a photograph, a video, a sound file, metadata associated with data already stored in other locations or metadata associated with data that cannot be stored as digital data.

6. The method of claim 1, wherein the receiving first and second brain activity information are acquired using a brain-computer interface (BCI), an electroencephalography (EEG), a functional magnetic resonance image (fMRI), or a magnetoencephalography (MEG).

7. The method of claim 1, wherein during acquisition of the second brain activity information, the user is engaged in thinking about the circumstances in which the data to be stored was generated or stored.

8. The method of claim 1, wherein during acquisition of the second brain activity information, the user is engaged in talking about, writing about, or engaging in a physical gesture related to the circumstances in which the data to be stored was generated or stored.

9. The method of claim 1, wherein brain activity is continuously received from the user and both the first brain activity and the second brain activity are portions of the continuously received brain activity.

10. A method for storing and recalling stored data, comprising:
receiving metadata associated with previously stored data, including a location of the data;
receiving brain activity information from a first user;
hashing the received brain activity and metadata to generate a first brain activity information hash value;
indexing the previously stored data according to the first brain activity information hash value;
receiving a set of thought attributes and metadata from a second user;
simulating brain activity information based on the received set of thought attributes;
hashing the simulated brain activity and metadata to generate a second brain activity information hash value;
identifying a location of the stored data by matching the second brain activity information hash value to the first brain activity information hash value; and
retrieving the stored data based on the identified location.

11. The method of claim 1, wherein a sensitivity of the stored data is determined based on the received brain activity information and the stored data is automatically characterized as public or private based on the determined sensitivity.

12. The method of claim 11, wherein determining the sensitivity of the stored data based on the received brain activity information includes interpreting the received brain activity to estimate a subject matter of thought or a sensitivity level of thought.

13. The method of claim 11, wherein when the stored data is characterized as private, credentials of the second user are used to verify that the second user is permitted to access the stored data prior to retrieving the stored data.

14. A computer system comprising:
a processor; and
a non-transitory, tangible, program storage medium, readable by the computer system, embodying a program of instructions executable by the processor to perform method steps for storing and recalling stored data, the method comprising:
receiving data to be stored
receiving first brain activity information from a user;
hashing the received first brain activity to generate a first brain activity information hash value;
storing the received data to be stored within a database; and
indexing the stored data, within the database, according to the first brain activity information hash value.

15. The computer system of claim 14, wherein the method further comprises:
receiving a request to recall the stored data;
receiving second brain activity information from a user;
hashing the received second brain activity to generate a second brain activity information hash value;
identifying a location of the stored data, within the database, based on the indexing, by matching the second brain activity information hash value to the first brain activity information hash value; and
retrieving the stored data based on the identified location.

16. The computer system of claim 14, wherein prior to storing the received data, environmental sensor data is received, the indexing of the stored data is based on both the first brain activity information hash value and the environmental sensor data.

17. The computer system of claim 16, wherein the environmental sensor data is acquired using a global positioning service (GPS) device, an accelerometer, a barometer, or one or more physiological sensors.

18. The computer system of claim 14, wherein a sensitivity of the stored data is determined based on the received first brain activity information and the stored data is automatically characterized as public or private based on the determined sensitivity.

19. The computer system of claim 18, wherein determining the sensitivity of the stored data based on the received first brain activity information includes interpreting the received first brain activity to estimate a subject matter of thought or a sensitivity level of thought.

20. The computer system of claim 18, wherein when the stored data is characterized as private, credentials of a second user are verified before the second user is permitted to access the stored data.
